(19) United States
(12) Patent Application Publication
(10) Pub. No.: US 2017/0266319 A1
(43) Pub. Date: Sep. 21, 2017

(54) METHODS AND COMPOSITIONS FOR TREATING CONDITIONS ASSOCIATED WITH SPINAL CORD INJURY

(71) Applicant: Children's Hospital Medical Center, Cincinnati, OH (US)

(72) Inventors: Masaki Ueno, Niigata (JP); Yutaka Yoshida, Cincinnati, OH (US); Phillip G. Popovich, Columbus, OH (US)

(21) Appl. No.: 15/464,471

(22) Filed: Mar. 21, 2017

Related U.S. Application Data
(60) Provisional application No. 62/310,860, filed on Mar. 21, 2016.

(51) Int. Cl. A61K 48/00; A61J 1/18; C12N 7/00; A61K 38/17; A61K 31/498; A61K 31/662

(52) U.S. Cl. CPC A61K 48/00; A61K 31/498; A61K 31/662; C12N 7/00; A61K 38/1787; A61J 1/18; C12N 2750/14143; C12N 2750/14132

(57) ABSTRACT

In one aspect, a method of treating a condition associated with spinal cord injury is disclosed, wherein an agent that inhibits excitatory neurons and/or activates inhibitory interneurons may be administered to an individual in need thereof. Articles of manufacture and kits related to the disclosed methods are also disclosed.

### CROSS REFERENCE TO RELATED APPLICATIONS

This application claims the benefit of and priority to U.S. Provisional Application Ser. No. 62/310,860 entitled “Neurogenic Method to Restore Immune Dysfunction after Spinal Cord Injury,” filed Mar. 21, 2016, the contents of which are incorporated by reference herein in their entirety and for all purposes.

### BACKGROUND

Spinal cord injury (SCI) at high spinal levels (e.g., above thoracic level 5) causes systemic immune suppression. However, the underlying mechanisms are unknown. In humans having SCI, various conditions may develop, including autonomic dysreflexia, a clinical syndrome marked by the sudden onset of potentially life-threatening high blood pressure, as well as immune suppression.

There is a need in the art for methods and compositions for the treatment of conditions associated with spinal cord injury. The instant disclosure addresses one or more of the aforementioned needs in the art.

### BRIEF SUMMARY

In one aspect, a method of treating a condition associated with spinal cord injury is disclosed, wherein an agent that inhibits excitatory neurons and/or activates inhibitory interneurons may be administered to an individual in need thereof. Articles of manufacture and kits related to the disclosed methods are also disclosed.

### BRIEF DESCRIPTION OF THE DRAWING

FIG. 1. Post-injury splenic atrophy and trans-synaptic labeling of spinal cord-spleen circuit in T3 SCI mice. (Panel A-E) SCI-dependent splenic atrophy caused by T3 SCI at 28 days post-injury. Gross anatomy of representative spleens from control and T3 SCI mice (Panel A), hematoxylin and eosin staining of spleen tissue sections (Panel B and Panel C), immunostaining of CD45R (B220; green), a marker of B cells, with DAPI (blue). Scale bars, 2 mm (Panel A); 500 μm (Panel B, Panel C); 100 μm (Panel D, Panel E). (Panel F-Panel Q). Horizontal sections of spinal cord in control (F-I), T9 SCI (J-M), and T3 SCI mice (N-Q), 96 hr after intrasplenic PRV injection. Images of 2 serial adjacent sections of T6-8 are shown, stained with anti-GFP antibody (green). Rostral is left. Magnified view of dotted squares in F, G, J, K, N, and O are shown in H, I, L, M, P and Q, respectively. Scale bars, 200 μm (f, g, j, k, n, o); 100 μm (h, i, l, m, p, q). (r-t). Number of PRV-positive cells was quantified in medial (r), intermediate (s), and lateral zone (t) of the spinal cord gray matter (see h) from T1 to S2 level then were compared between control (blue bars), T9 SCI (green bars), and T3 SCI mice (red bars). Dark bars, ipsilateral side; light bars, contralateral side of the spinal cord. n=4-5, One way ANOVA followed by Tukey-Kramer test, *p<0.05.

FIG. 2. Identification of spinal interneurons in the spinal cord-splenic sympathetic circuit. (a-l) Representative images of GFP (PRV, green), Chat (red), and FG (blue) labeled neurons at T6 spinal level in control (a-d), T9 SCI (e-h), and T3 SCI mice (i-l) after double-labeling with FG (i.p.) and PRV (intrasplenic). Horizontal spinal cord sections are oriented with rostral edge of spinal cord to the left. Dotted boxes in left panels are magnified in individual channels to the right. Arrows indicate FG⁻/PRV⁺ spinal interneurons. Scale bar, 50 (m) Quantification of the ratio of spinal interneurons (FG⁻/PRV⁺) to SPNs (FG⁺/PRV⁺). Control, n=4; T9 SCI, n=4; T3 SCI, n=4; One way ANOVA followed by Tukey-Kramer test, *p<0.05. (n-y) Neurotransmitter subtype of spinal interneurons in T3 SCI. (n-q) GFP (PRV, green), Chat (red), and FG (blue). (r-u) RFP (PRV, red), GFP (Vglut2, green), and FG (blue) in Vglut2-Cre; cc-EGFP mice. (v-y) RFP (PRV, red), GFP (Vgat, green), and FG (blue) in Vgat-Cre; cc-EGFP mice. Arrows indicate double-positive interneurons (FG⁻). (z) The ratio of Chat⁺, Vglut2-GFP⁺ and Vgat-GFP⁺ in FG⁻/PRV⁺ spinal interneurons in T3 SCI mice (n=3).

FIG. 3. Chemogenetic silencing of spinal interneurons rescues immune cells after T3 SCI. (a) Design of hM4Di-mCherry-expressing AAV, using FLEX switch strategy to induce Cre-mediated transgene inversion and expression in the thoracic spinal cord. (b) Experimental time course. (c) Image of spleens from individual mice at day 28 after T3 SCI of wild-type (no Cre-expression), Vglut2-Cre and Chat-Cre mice injected with AAV-hSyn-DIO-hM4Di-mCherry and CNO injection. (d) Wet spleen weights at day 28 post-injury. WT control, n=9; WT+T3 SCI, n=8; WT+AAV-hSyn-DIO-hM4DimCherry+T3 SCI+CNO, n=8; Vglut2-Cre+AAV-hSyn-DIO-hM4Di-mCherry+T3 SCI+CNO, n=8; Chat-Cre+AAV-hSyn-DIO-hM4Di-mCherry+T3 SCI+CNO, n=8; One way ANOVA followed by Tukey-Kramer test, *p<0.05, **p<0.01. (e) The number of total splenocytes. (f-h) The number of B220⁺ B cells (0, CD4⁺ (g) and CD8⁺ T cells (h). WT control, n=9; WT+T3 SCI, n=8; WT+AAV-hSyn-DIO-hM4DimCherry+T3 SCI+CNO, n=7; Vglut2-Cre+AAV-hSyn-DIO-hM4Di-mCherry+T3 SCI+CNO, n=7; Chat-Cre+AAV-hSyn-DIO-hM4Di-mCherry+T3 SCI+CNO, n=5; One way ANOVA followed by Tukey-Kramer test, *p<0.05, **p<0.01.

FIG. 4. Distribution of PRV-positive cells throughout the central nervous system (CNS) that are directly or indirectly connected to the spleen, i.e., a neural-splenic circuit. (a and b) Post-ganglionic adrenergic sympathetic nerves innervate the spleen. PACT clearing of spleen (a) and staining with anti-tyrosine hydroxylase (TH) antibody (b, red). TH⁺ fibers broadly innervate the spleen, which allows PRV to be transported retrogradely into sympathetic chain ganglia and then into the CNS. Scale bar, 2 mm. (c) Schema of retrograde and transsynaptic labeling of neural-splenic circuit by PRV. (d) Distribution summary of PRV-GFP positive cells in the CNS, 72-144 hours after PRV injection into the spleen (modified from⁸). Time points when PRV-GFP⁺ cells first appeared are indicated as black to white colors in each CNS region. (e-x) Representative images of PRV-GFP⁺ cells in different CNS regions and time points after PRV152 injection into the spleen. Mice were perfused at 48 (day 2, n=2), 72 (day 3, n=2), 96 (day 4, n=2), 108 (day 4.5, n=1), 120 (day 5, n=2), 144 (day 6, n=3), and 168 hr (day 7, n=2) after injection. To verify that PRV labeling from the spleen occurs via the splenic nerve and is not the result of spurious labeling due to blood-borne virus, the splenic nerve was cut before PRV injection then analyzed at 120 hr (n=2). Thoracic cord (T4-8), e (72 hr), f (96 hr), g (120 hr); dorsal motor nucleus of vagus (10N), h (108 hr), i (120 hr), j (120 hr, splenic nerve cut); Reticular formation, k (120 hr), l (144 hr), m (120 hr, splenic nerve cut); paraventricular nucleus (PVN), n (120 hr), o (144 hr), p (120 hr, splenic nerve cut); PsTh, q (120 hr), r (144 hr); zona incerta (ZI), s (120 hr), t (144 hr); locus coeruleus (LC), u (144 hr); periaqueductal gray (PAG), v (144 hr); basolateral amygdaloid nucleus (BLP), w (144 hr); bed nucleus of the stria terminalis (BST), x (144 hr). Coronal sections. Scale bar, 200 μm.

FIG. 5. PRV⁺ spinal sympathetic neurons innervating the spleen in control and SCI mice. (a-i) Horizontal sections of spinal cord in control (a-c), T9 SCI (d-f), and T3 SCI mice (g-i), 96 hrs after PRV injection into the spleen. Stained with anti-GFP antibody (green). T2-4 (a, d, g), T9-11 (b, e, h), and L5-S1 level (c, f, i). Arrowheads, lesion sites (e and g). Rostral is left. Scale bar, 200 μm. (j-m) Distribution of PRV⁺ cells in dorso-ventral axis of thoracic cord after T3 SCI. (j) Schematic figure of transverse section of thoracic cord. Dotted lines represent approximate dorsoventral positions of images shown in k, l, m, and FIGS. 1n-q. (k-m) Horizontal images of PRV-GFP⁺ cells distributed in dorsoventral axis. Rostral is left. Scale bar, 100 μm. (n-q) Representative images of Vglut2-GFP⁺ (green)/PRV⁺ (RFP, red) neurons in the sacral spinal cord after T3 SCI. Right panels (o-q) are higher magnification of the neurons indicated by arrows in (n). 76.7±2.86% of PRV⁺ were Vglut2-GFP⁺ neurons at the sacral level (n=2). Scale bars, 100 μm (n), 50 μm (o-q).

FIG. 6. Time-dependent increase in number of VGlut2⁺ pre-synaptic contacts on SPNs after T3 SCI. (a) Schematic illustrating intrasplenic injections of PRV and intraperitoneal injections of FG to distinguish SPNs (FG⁺/PRV⁺) from interneurons (FG⁻/PRV⁺) in spinal cord-splenic circuit. (b-g) Representative confocal images of FG⁺ (blue)/PRV⁺ (green) SPNs and vGlut2 puncta (red) in control (b, c) and at 10 (d, e) and 28 days post-injury (f, g). SPNs in (b), (d) and (f) (arrowheads) are reconstructed into 3D images using Imaris software are isolated in (c), (e) and (g), respectively. Scale bar, 40 μm. (f) Quantification of the density of vGlut2⁺ synaptic puncta on FG⁺/PRV⁺ SPNs at day 7-10 and day 28, the time points before and after onset of immune suppression in T3 SCI mice′. Dotted line indicates the density of vGlut2⁺ synaptic puncta quantified from naive/uninjured mice (n=2 mice, 44 total neurons) and is provided as a reference for post-injury changes in puncta density. *p<0.05, Student's t test. Day 7, n=3; day 10, n=1; day 28, n=5.

FIG. 7. Activating visceral-sympathetic reflexes via CRD induces c-Fos expression in vGlut2⁺ excitatory spinal interneurons. (a and b) CRD induced c-Fos expression (red) in the thoracic cord of T3 SCI mice. T6-8 level, horizontal sections. T3 SCI without (a) and with CRD (b) at day 28. (c) Quantitative analyses of c-Fos⁺ cells at T4-9 level of control or T3 SCI mice with or without CRD. n=3-5; One way ANOVA followed by Tukey-Kramer test, **p<0.01. (d-f) c-Fos expression (red) in GFP⁺ cells (green) in Vglut2-Cre; cc-GFP mice with T3 SCI and CRD at day 28 (arrows). Scale bars, 200 μm (a, b); 50 (d-f). (g) Quantification of Vglut2-GFP⁺ and Chat-GFP⁺ population in cFos-expressing cells with T3 SCI and CRD at day 28 (n=3).

FIG. 8. Introduction of hM4Di in spinal interneurons by AAV-hSyn-DIO-hM4Di-mCherry. (a) hM4Di-mCherry expression in Vglut2-Cre mice. T6-8 level at day 28 after T3 SCI. (b-d) hM4Di-mCherry (red) is expressed in GFP⁺ neurons in Vglut2-Cre; cc-GFP mice (arrows). (e) hM4Di-mCherry expression in Chat-Cre mice. T6-8 level at day 28 after T3 SCI. (f-h) hM4Di-mCherry (red) is expressed in GFP⁺ neurons in Chat-Cre; cc-GFP mice (arrows). Note that most hM4Di-mCherry⁺ cells are located in the intermediate and medial zone. Few exist in the lateral zone where SPNs predominate. Horizontal sections. Scale bars, 500 μm (a, e); 50 μm (b-d, f-h). (i) Percentage of hM4Di-mCherry⁺/GFP⁺ cells in total hM4Di-mCherry⁺ cells in Vglut2-Cre; cc-GFP and Chat-Cre; cc-GFP mice. (j) A representative image of brain stem region (reticular formation) in Vglut2-Cre mice at day 28 after T3 SCI, injected with AAV-hSyn-DIO-hM4Di-mCherry at thoracic level at P14. No hM4Di-mCherry was visible in any sections indicating that the AAV vector does not get retrogradely transported into the brain stem. Thus, no descending modulation by brainstem circuitry can contribute to hM4Di-mediated inhibition of spinal circuitry. Scale bar, 200 μm.

FIG. 9. Chemogenetic silencing of spinal autonomic circuitry reduces c-Fos expression elicited by colorectal distension (CRD), a potent visceral-sympathetic reflex stimulus. (a-f) c-Fos expression in horizontal thoracic spinal cord sections of AAV-hSyn-DIO-hM4Di-mCherry-injected T3 SCI Vglut2-Cre mice injected with vehicle (a-c) or CNO and with CRD (d-f). Scale bar, 50 μm (g, h) Quantification of density of c-Fos⁺ cells and percentage of c-Fos⁺/hM4Di-mCherry⁺ cells. *p<0.05, Student's t test, n 3.

FIG. 10. Representative flow cytometry scatter plots of splenocytes derived from control and SCI mice with or without chemogenetic manipulation of autonomic circuitry. (a, c, e, g, i) Gating strategies using FSC-A vs SSC-A, FSC-H vs FSC-W and SSC-H vs SSC-W plots (to remove doublets). (b, d, f, h, j) Dot plots of B220, CD4, and CD8-labeled lymphocytes from WT control (b), WT+T3 SCI (d), WT+AAV-hSyn-DIO-hM4Di-mCherry+T3 SCI+CNO (f), Vglut2-Cre+AAV-hSyn-DIO-hM4Di-mCherry+T3 SCI+CNO (h), and Chat-Cre+AAV-hSyn-DIO-hM4Di-mCherry group (j). Mean percentage of B220⁺, CD4⁺ or CD8⁺ cells generated by gating for each cell population was multiplied by total number of splenocytes to generate bar graphs in FIGS. 3f-h.

FIG. 11. Proposed model by which activation of a sympathetic anti-inflammatory reflex (SAR) causes immune suppression after high-level SCI. (a) Normally, there is tonic brainstem control over sympathetic preganglionic neurons (SPNs). This tonic regulation is essential for normal neural-immune communication and maintenance of homeostasis. (b) After high-level T3 SCI (red dotted lines), supraspinal control over SPNs is lost allowing normally innocuous stimuli below the level of injury to elicit exaggerated and potentially life-threatening autonomic reflexes (gray dotted lines). New data show that profound plasticity develops within spinal autonomic circuitry below the level of SCI. Notably, new connections form between SPNs and spinal excitatory interneurons throughout and beyond the thoracic spinal cord (FIG. 5, i and n-q). This rewired circuit is the neural substrate underlying SAR-mediated suppression of immune function.

FIG. 12. Proposed model by which activation of a sympathetic anti-inflammatory reflex (SAR) causes immune suppression after high-level SCI. (a) Normally, there is tonic brainstem control over sympathetic preganglionic neurons (SPNs). This tonic regulation is essential for normal neural-immune communication and maintenance of homeostasis. (b) After high-level T3 SCI (red dotted lines), supraspinal control over SPNs is lost allowing normally innocuous stimuli below the level of injury to elicit exaggerated and potentially life-threatening autonomic reflexes (gray dotted lines). New data show that profound plasticity develops within spinal autonomic circuitry below the level of SCI. Notably, new connections form between SPNs and spinal excitatory interneurons throughout and beyond the thoracic spinal cord (FIG. 6, i and n-q). This rewired circuit is the neural substrate underlying SAR-mediated suppression of immune function.

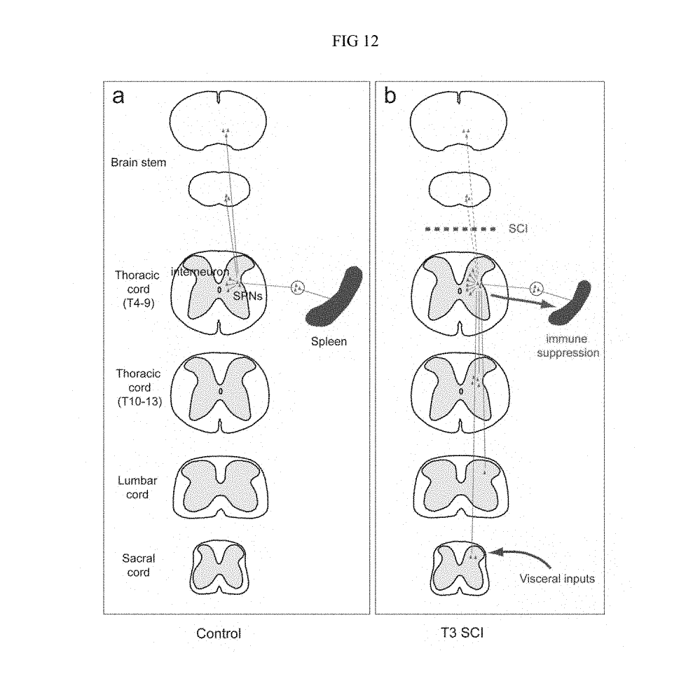

### DETAILED DESCRIPTION

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms and expressions used herein have the ordinary meaning as is accorded to such terms and expressions with respect to their corresponding respective areas of inquiry and study except where specific meanings have otherwise been set forth herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

“Therapeutically effective amount” relates to the amount or dose of an active compound or composition described herein that will lead to one or more therapeutic effect, in particular, desired beneficial effects. A therapeutically effective amount of a substance can vary according to factors such as the disease state, age, sex, and weight of the subject, and the ability of the substance to elicit a desired response in the subject. Dosage regime may be adjusted to provide the optimum therapeutic response. For example, several divided doses may be administered daily or the dose may be proportionally reduced as indicated by the exigencies of the therapeutic situation.

The phrase “pharmaceutically acceptable,” as used in connection with compositions of the disclosure, refers to molecular entities and other ingredients of such compositions that are physiologically tolerable and do not typically produce untoward reactions when administered to a subject (e.g., human). In certain embodiments, as used herein, the term “pharmaceutically acceptable” means approved by a regulatory agency of a Federal or a state government or listed in the U.S. Pharmacopeia or other generally recognized pharmacopeia for use in mammals (e.g., humans).

The term “carrier” applied to pharmaceutical compositions of the disclosure refers to a diluent, excipient, or vehicle with which an active compound (e.g., dextromethorphan) is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like. Suitable pharmaceutical carriers are described in “Remington's Pharmaceutical Sciences” by E. W. Martin, 18th Edition.

Spinal cord injury (SCI) at high spinal levels (e.g., above thoracic level 5) causes systemic immune suppression. However, the underlying mechanisms are unknown. Applicant has found that profound plasticity develops within spinal autonomic circuitry below the injury, creating a sympathetic anti-inflammatory reflex, and that chemogenetic silencing of this reflex circuitry blocks post-SCI immune suppression. Novel therapeutic options for limiting the devastating consequences of post-traumatic autonomic hyperreflexia and post-injury immune suppression are disclosed.

Spinal cord injury (SCI) at or above the T5 spinal level disrupts tonic brain stem control over the spinal autonomic circuitry. Consequently, normally innocuous stimuli below the level of injury, such as filling of bladder or bowel, elicit uncontrolled, exaggerated and potentially life-threatening reflex activation of autonomic circuitry including sympathetic preganglionic neurons (SPNs) and post-ganglionic noradrenergic (norepinephrine; NE) fibers that innervate vasculature, viscera and lymphoid tissues. In SCI mice and humans, eliciting a visceral-sympathetic reflex can provoke autonomic dysreflexia (AD), a clinical syndrome marked by the sudden onset of potentially life-threatening high blood pressure^{1, 2}. Recently, Applicant found that eliciting AD also triggers immune suppression³⁻⁵.

Spontaneous and episodic activation of this circuitry potentially explains why people with high-level SCI become more susceptible to infection—a leading cause of morbidity and mortality after SCI⁶. However, the neuronal substrates responsible for immune suppressive autonomic reflexes after high-level SCI have not been determined. Injuries at the T3 and T9 spinal levels remove or preserve, respectively, descending brainstem control over SPNs, and only after T3 SCI does sympathetic hyperreflexia develop with concomitant splenic leukocyte depletion and immune suppression^{3, 4}.

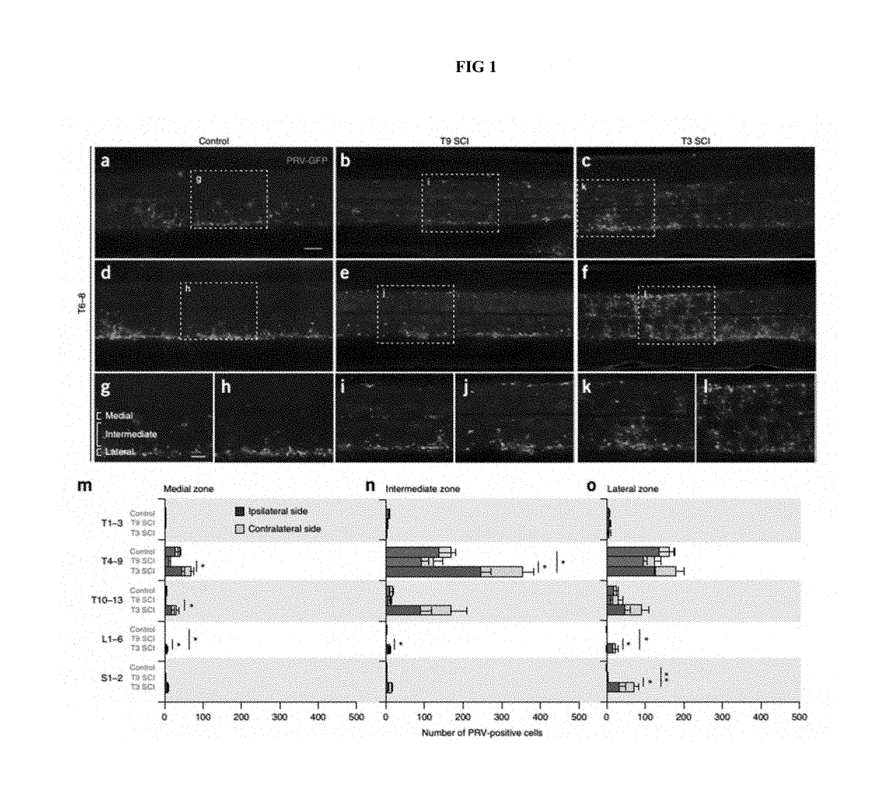

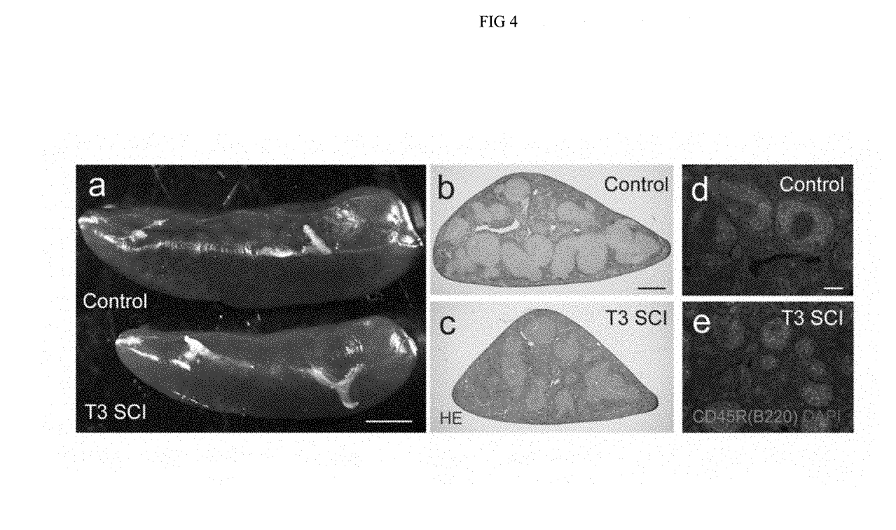

Accordingly, Applicant first determined whether injuries at the T3 and T9 spinal levels differentially affect the neuroanatomical substrate that connects spinal cord autonomic neurons with the spleen, a major secondary lymphoid organ. Consistent with previous reports³, T3 but not T9 SCI caused profound splenic atrophy and leucopenia (only T3 SCI shown in FIGS. 1a-e). To map the circuitry connecting spinal cord to spleen, trans-synaptic neurotropic retrograde GFP-expressing pseudorabies virus (PRV; PRV152⁷) was injected into the spleen of uninjured (control) or SCI mice 28 days after receiving a SCI at the T3 or T9 spinal level. (FIG. 1, A-E). In control mice, PRV-GFP⁺ cells were identified then mapped throughout the spinal cord and brain (FIG. 1, F-I, FIG. 4 and TABLE 1).

TABLE 1
Temporal Distribution of PRV-Infected Neurons

| | 48 hr | 72 hr | 96 hr | 108 hr | 120 hr | 144 hr | 168 hr |
|---|---|---|---|---|---|---|---|
| Area | (n = 2) | (n = 2) | (n = 2) | (n = 1) | (n = 2) | (n = 3) | (n = 2) |
| Spinal cord | | | | | | | |
| T1-3 | (n.e.) | (n.e.) | −~± | (n.e.) | (n.e.) | (n.e.) | dead |
| T4-9 | − | ±~+ | + | + | + (d) | + (d) | |
| T10-12 | (n.e.) | (n.e.) | −~± | − | (n.e.) | (n.e.) | |
| Brain | | | | | | | |
| dorsal motor nucleus of vagus (10N) | − | − | − | ++ | ++ (d) | ++ (d) | |
| reticular formation | − | − | − | − | ±~+ | ++ | |
| locus coeruleus (LC) | − | − | − | − | −~± | ++ | |
| subcoeruleus nucleus (SubC) | − | − | − | − | −~± | ++ | |
| periaqueductal gray (PAG) | − | − | − | − | − | + | |
| parasubthalamic nucleus (PsTh) | − | − | − | − | ±~+ | ++ | |
| zona incerta (ZI) | − | − | − | − | ±~+ | ++ | |
| dorsomedial hypothalamus (DM) | − | − | − | − | − | + | |
| paraventricular nucleus (PVN) | − | − | − | (n.e.) | ±~+ | ++ | |
| basolateral amygdaloid nucleus (BLP) | − | − | − | − | − | ++ | |
| bed nucleus of the stria terminalis (BST) | − | − | − | − | − | ++ | |

−, not detected;
±, a few positive cells;
+, moderate number of positive cells;
++: numerous positive cells;
(d), neurons start degenerating;
(n.e.), not examined;
dead, mice were dead

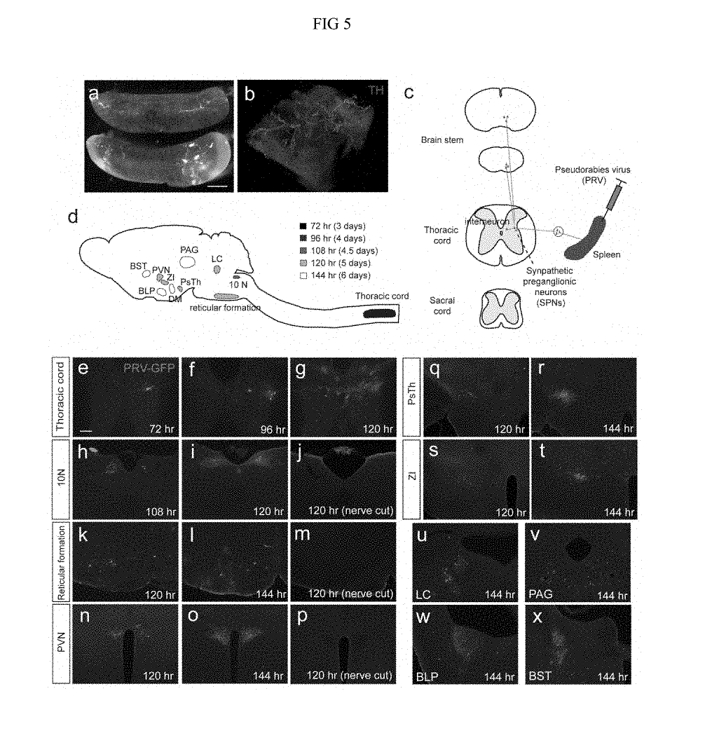

In control spinal cords, most PRV-GFP⁺ cells were found ipsilateral to the site of intrasplenic injection (left side) within the lateral and intermediate gray matter at T4-9 spinal levels (FIGS. 1f-i, r-t). Such labeling is consistent with the location of the sympathetic preganglionic neurons (SPNs) previously shown to innervate the spleen in rats⁸. PRV-GFP⁺ cells were found in similar numbers and distribution in control mice and mice with T9 SCI (FIGS. 1j-m, r-t). Conversely, although total numbers of PRV-GFP⁺ cells within the lateral zone of the T4-9 segment were similar across the groups, the number nearly doubled in the intermediate and medial zones after T3 SCI (FIGS. 1n-q, r-t). These differences were further exaggerated between T3 SCI and control groups at remote caudal spinal levels including sacral spinal cord (FIG. 1, Panels R-T, FIG. 5).

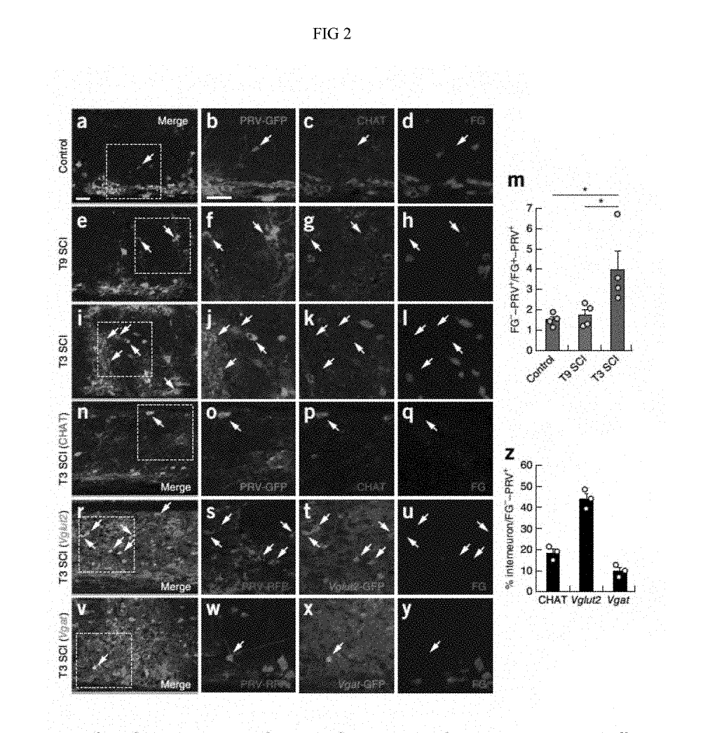

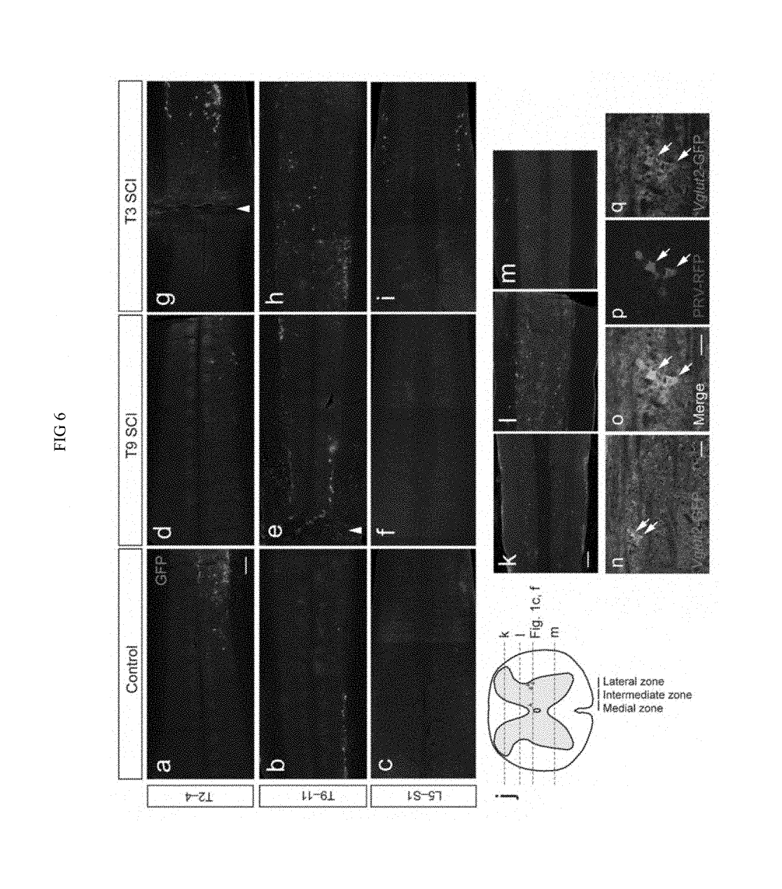

The GFP⁺ neurons that become visible in the medial and intermediate zones after T3 SCI are likely interneurons that form new synapses with SPNs. To selectively label SPNs and distinguish them from trans-synaptically-labeled PRV-GFP⁺ interneurons, fluorogold (FG), a retrograde tracer, was injected into the intraperitoneal cavity (FIG. 6, Panel A)^{8, 9}. SPNs of the splenic circuit were identified as FG/PRV-GFP-double-positive (FG⁺/PRV⁺) cells, whereas interneurons do not label with FG and are FG⁻/PRV⁺. In control spinal cord, FG⁺/PRV⁺ neurons were predominantly found along the lateral edge of the gray matter and consistent with their motor neuron lineage, FG⁺ SPNs were choline acetyltransferase-positive (Chat⁺) (FIG. 2a-d). In control mice, few FG⁻/PRV⁺ interneurons were in the intermediate or medial zones (FIGS. 2a-d, arrows). Labeling distribution and cell densities were similar in T9 SCI and control mice (FIG. 2e-h). However, after T3 SCI, more than twice as many FG⁻/PRV⁺ interneurons were found in the intermediate and medial zones (FIG. 2i-l (arrows), FIG. 2m). These data indicate that the enhanced connectivity that develops in autonomic circuits after high-level SCI is mainly caused by plasticity within spinal interneuron pools connecting with SPNs.

Referring to the phenotype of these spinal interneurons, Chat immunostaining was used to label cholinergic neurons whereas glutamatergic excitatory or GABAergic/glycinergic inhibitory neurons were genetically labeled using Vglut2-Cre¹⁰; CAG-lox-CAT-lox-EGFP (cc-EGFP¹¹) or in Vgat-Cre¹⁰; cc-EGFP mice, respectively. Most FG⁻/PRV⁺ interneurons were Vglut2⁺ (44.0±2.65% in T3 SCI; 29.2±1.75% in controls; FIG. 5, n-q) while fewer were Chat⁺ (18.3±2.15% in T3 SCI; 47.1±6.45% in controls) or Vgat⁺ (9.88±1.86% in T3 SCI; 16.1±0.87% in controls) (FIGS. 2n-z). These data suggest that Vglut2⁺ interneurons are preferentially involved in level-dependent plasticity within the autonomic circuitry after T3 SCI. Consistent with this conclusion, Applicant observed a time-dependent increase in the number of Vglut2⁺ pre-synaptic puncta that contact FG⁺/PRV⁺ SPNs (FIG. 6). This suggests an increase in connectivity between excitatory interneurons and SPNs after T3 SCI.

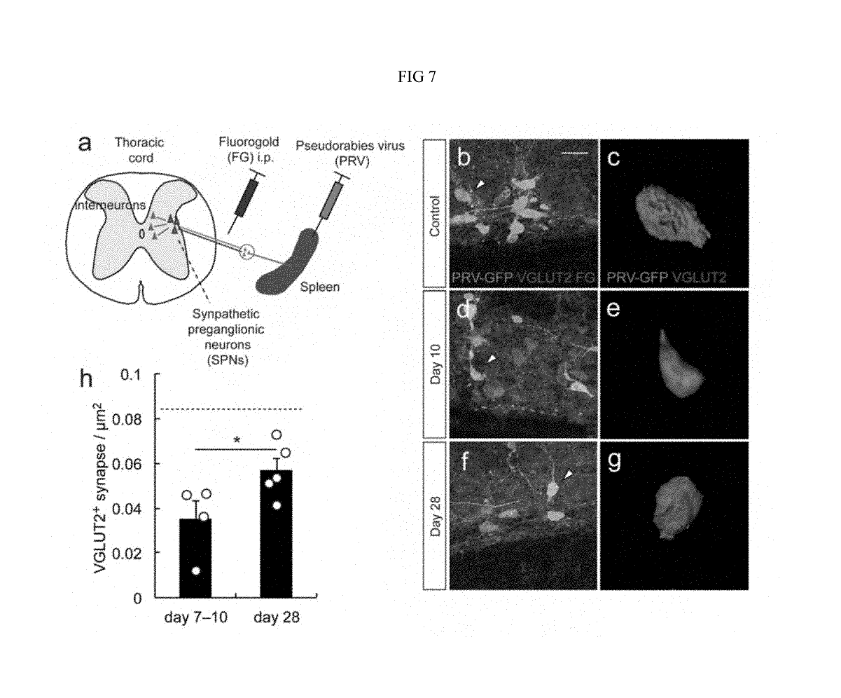

Without intending to be limited by theory, the post-injury integration of large numbers of glutamatergic interneurons into the spinal-splenic circuitry may explain why spontaneous activation of visceral-sympathetic reflexes (e.g., filling of bladder or bowel), becomes exaggerated as a function of time post-SCI and causes chronic immune suppression³. To determine whether the augmented interneuronal structural plasticity is associated with an increase in sensitivity to visceral-sympathetic reflex activation, expression of c-Fos, an activity-dependent immediate early gene, was quantified in mice with high-level SCI after eliciting colorectal distension (CRD). Similar to what has been reported in spinal injured rats^{12, 13} the total number of c-Fos⁺ neurons increased throughout the intermediate and medial gray matter of SCI mice after CRD (FIG. 7, a-c). Most c-Fos⁺ cells (>55%) were Vglut2⁺ interneurons while <20% were Chat⁺ (FIG. 7, d-g).

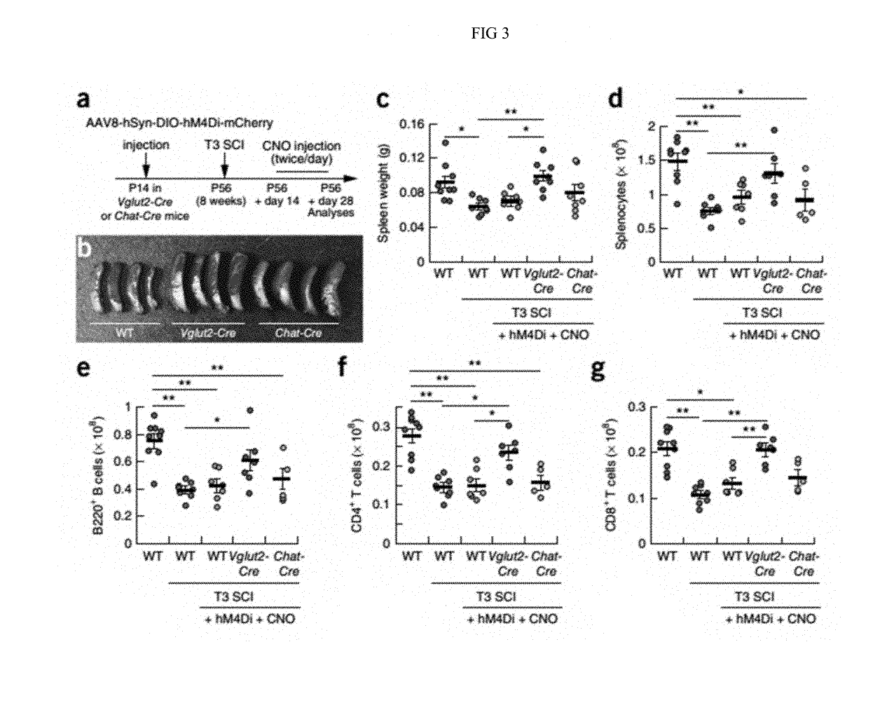

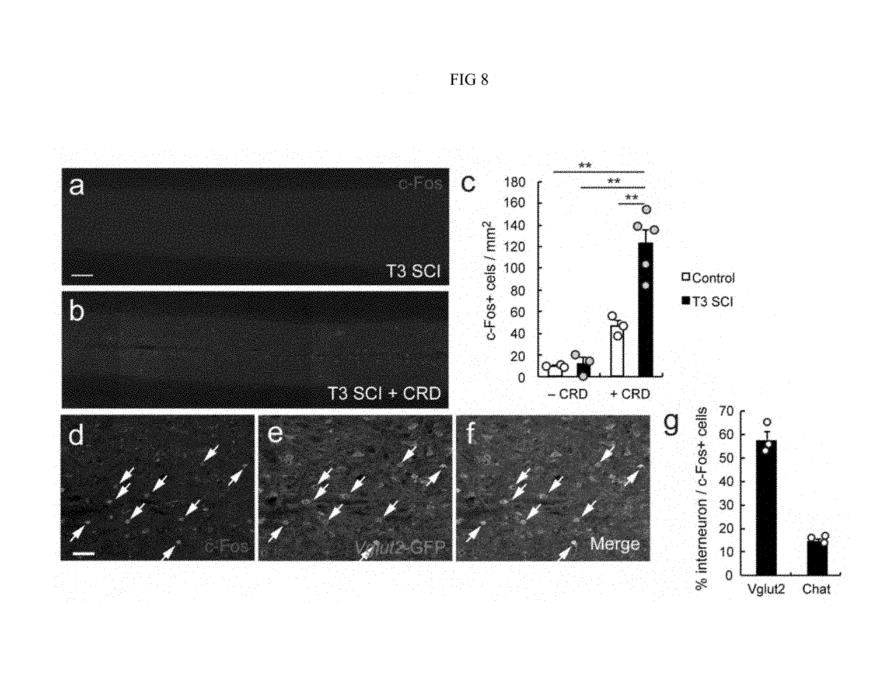

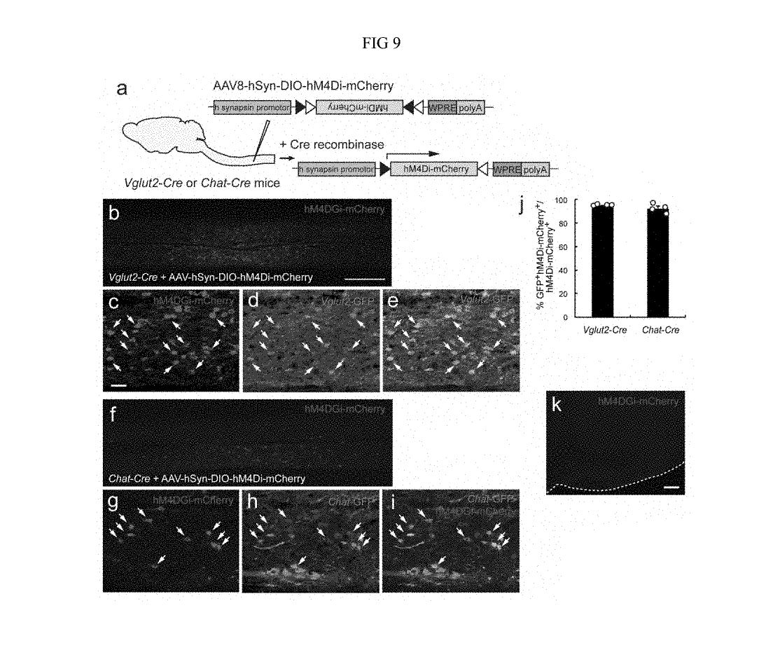

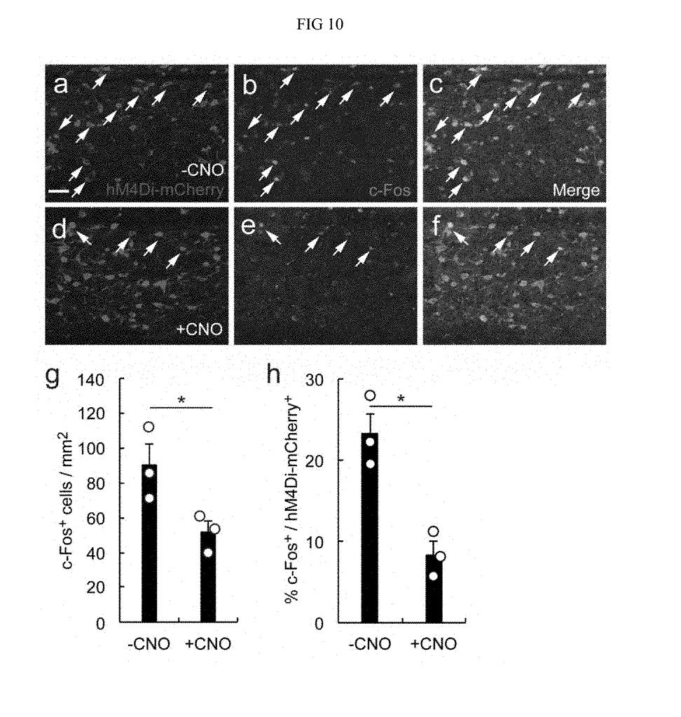

After T3 SCI, both spontaneous and intentional activation of spinal autonomic reflexes cause leukocyte apoptosis and immune suppression³. To prove a causal role for Vglut2⁺ or Chat⁺ spinal interneurons in propagating an immune suppressive autonomic reflex, Applicant silenced these neurons using chemogenetics¹⁴. Specifically, an engineered Gi/o-coupled human muscarinic M4 designer receptor exclusively activated by a designer drug (hM4Di DREADD) was expressed in Vglut2⁺ or Chat⁺ neurons using an adeno-associated viral (AAV) vector. Neuronal activity is then selectively inhibited in these cells following systemic injection of clozapine-N-oxide (CNO)¹⁴ (FIG. 3a). To target these interneurons, AAV8-hSyn-DIO-hM4D(Gi)-mCherry (DIO=“double-floxed inverted orientation) was injected into intermediate thoracic gray matter of Vglut2-cre or Chat-cre mice at P14 to express hM4Di in a Cre-dependent manner (FIGS. 3a, b, FIG. 8). Six weeks later (P56), all mice received T3 SCI and then to silence neurons, CNO (1 mg/kg) was injected 2×/day from day 14-27 post-injury (FIG. 3b), when exaggerated immune suppressive reflexes develop spontaneously in SCI mice³. The data reveal significant silencing of hM4DGi⁺ spinal neurons (FIG. 9) with immune protection only in T3 SCI Vglut2-Cre mice injected with AAV8-hSyn-DIO-hM4D(Gi)-mCherry and CNO. Splenic atrophy was reversed and total numbers of splenocytes, including CD4⁺ and CD8⁺ T cells and B220⁺ B cells, were restored to pre-injury levels (FIGS. 3c-h, FIG. 10). Although AAV8-hSyn-DIO-hM4D(Gi)-mCherry was effectively introduced into Chat⁺ interneurons, fewer Chat⁺ SPNs in the lateral zone were positive for hM4Di-mCherry as compared with interneurons located in the medial or intermediate zones (medial, 38.6±6.26%; intermediate, 55.5±5.17%; lateral, 5.86±1.54%, n=4: FIG. 8, e-h). Accordingly, only modest non-significant immune protection occurred in Chat-Cre mice (FIGS. 3c, d).

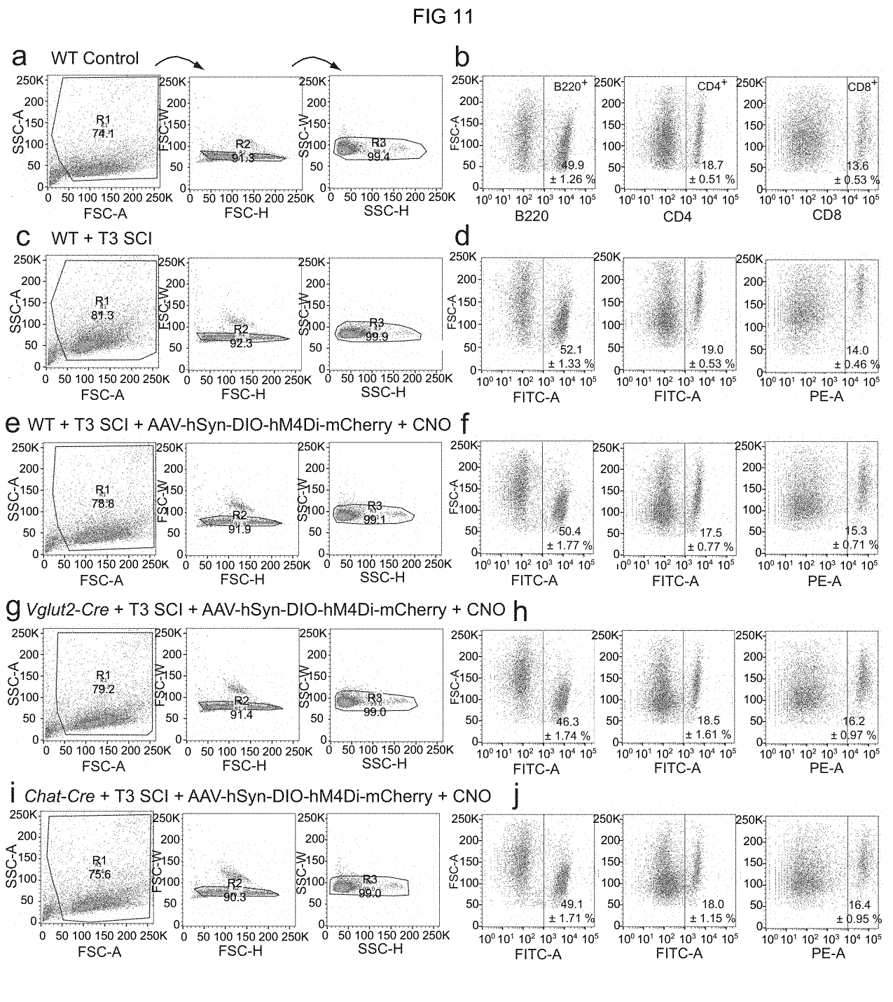

Thus, a novel neurogenic mechanism underlying immune suppression caused by high-level SCI has been identified by Applicant. Specifically, retrograde trans-synaptic labeling from the spleen of SCI mice reveals the formation of new and complex intraspinal circuitry, presumably due to ongoing plasticity and synaptogenesis within the spinal cord between primary sensory afferents, interneurons and SPNs. The receptive field for activating this newly formed circuit expands beyond the thoracic spinal segments that normally innervate secondary lymphoid tissues in naive/uninjured mice. As a result, after SCI, the uncontrolled spontaneous activation of spinal interneurons by visceral afferents (e.g., from bladder or bowel) causes exaggerated activation of SPNs and post-ganglionic noradrenergic fibers that innervate vasculature, viscera and lymphoid tissues (FIG. 11). Engaging this circuitry activates a sympathetic anti-inflammatory reflex (SAR) that causes chronic immune suppression. While the effects of SAR can be mitigated by repeat injections of adrenergic receptor antagonists³, Applicant has now shown that immune protection can be achieved by “silencing” aberrant intraspinal circuitry. In humans, high-level SCI elicits spinal cord injury-induced immune depression syndrome (SCI-IDS), a level-dependent neurogenic mechanism of immune suppression that increases the risk of developing infection (e.g., pneumonia)¹⁵. Thus, the development and application of similar techniques in humans could eliminate SCI-IDS and significantly improve quality of life for those living with SCI. Combinatorial approaches that silence the SAR in combination with systemic injection(s) of agents that boost hematopoiesis and stimulate immune function may be used.

In one aspect, a method of treating a condition associated with spinal cord injury is disclosed. The method may comprise the step of administering an agent that inhibits excitatory neurons and/or activates inhibitory interneurons to an individual in need thereof.

In one aspect, the condition may be selected from AD (autonomic dysreflexia), immune suppression, leukocyte apoptosis, splenic atrophy and/or leucopenia.

In one aspect, the agent that inhibits excitatory neurons and/or activates inhibitory interneurons may be an NMDA (glutamate receptor) antagonist or a pharmaceutically acceptable salt thereof.

In one aspect, the agent may be an a-amino-3-hydroxy-5-methyl-4-isoxazolepro-pionic acid (AMPA) receptor antagonist, or a pharmaceutically acceptable salt thereof.

In one aspect, the agent may be an antagonist having the structure of 6-cyano-7-nitroquinoxa-line-2,3-dione (CNQX), or a pharmaceutically acceptable salt thereof.

In one aspect, the agent may be an NMDA receptor antagonist DL-2-amino-5-phosphonovalerate (DL-APV), or a pharmaceutically acceptable salt thereof.

In one aspect, the agent may be DL-2-amino-4-phosphonobutyric acid (DL-APB), or a pharmaceutically acceptable salt thereof.

In one aspect, the agent may be selected from phencyclidine (PCP), dextromethorphan, ketamine, or a pharmaceutical salt thereof, and combinations thereof.

In one aspect, the agent may be selected from benzodiazepine and THC.

In one aspect, the agent may be a AAV-vGlut2-promoter-DREADD construct.

In one aspect, an article of manufacture is disclosed. The article of manufacture may comprise a container comprising a label; and a composition comprising an agent that inhibits excitatory neurons and/or activates inhibitory interneurons, wherein the label indicates that the composition is to be administered to an individual having a condition associated with a spinal cord injury, for example, wherein the agent is any one or combination of agents listed above.

In one aspect, a kit comprising an agent that inhibits excitatory neurons and/or activates inhibitory interneurons and instructions for treating a subject with a condition associated with spinal cord injury with said agent is disclosed. The kit may comprise a means for delivery of one or more aforementioned compositions to an individual in need thereof. In one aspect, the kit may be formulated for intravenous administration to an individual.

Dosage

As will be apparent to those skilled in the art, dosages outside of these disclosed ranges may be administered in some cases. Further, it is noted that the ordinary skilled clinician or treating physician will know how and when to interrupt, adjust, or terminate therapy in consideration of individual patient response.

In certain embodiment, the dosage of an agent disclosed herein, based on weight of the active compound, administered to prevent, treat, manage, or ameliorate pain in a subject may be about 0.25 mg/kg, 0.5 mg/kg, 0.1 mg/kg, 1 mg/kg, 2 mg/kg, 3 mg/kg, 4 mg/kg, 5 mg/kg, 6 mg/kg, or more of a subject's body weight. In another embodiment, the dosage of an agent disclosed herein to prevent, treat, manage, or ameliorate pain in a subject is a unit dose of about 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In one aspect, an agent disclosed herein may be present in an amount of from about 0.5% to about 95%, or from about 1% to about 90%, or from about 2% to about 85%, or from about 3% to about 80%, or from about 4%, about 75%, or from about 5% to about 70%, or from about 6%, about 65%, or from about 7% to about 60%, or from about 8% to about 55%, or from about 9% to about 50%, or from about 10% to about 40%, by weight of the composition.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms all utilizing dosage forms well known to those of ordinary skill in the pharmaceutical arts. The compositions may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A dosage regimen will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of pain can be readily determined by an ordinarily skilled physician

The pharmaceutical compositions may include suitable dosage forms for oral, parenteral (including subcutaneous, intramuscular, intradermal and intravenous), transdermal, sublingual, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. Oral preparations include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. For topical or nasal administration, penetrants or permeation agents that are appropriate to the particular barrier to be permeated are used in the formulation. Such penetrants are generally known in the art. The pharmaceutical compositions are prepared by conventional techniques appropriate to the desired preparation containing appropriate amounts of the active ingredient, that is, one or more of the disclosed active agents or a pharmaceutically acceptable salt thereof according to the invention.

The dosage of an agent disclosed herein used to achieve a therapeutic effect will depend not only on such factors as the age, weight and sex of the patient and mode of administration, but also on the degree of inhibition desired and the potency of an agent disclosed herein for the particular disorder or disease concerned. It is also contemplated that the treatment and dosage of an agent disclosed herein may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

Kits

Kits are also provided. In one aspect, a kit may comprise or consist essentially of agents or compositions described herein. The kit may be a package that houses a container which may contain a composition comprising an oxime or pharmaceutically acceptable salt thereof as disclosed herein, and also houses instructions for administering the agent or composition to a subject. In one aspect, a pharmaceutical pack or kit is provided comprising one or more containers filled with one or more composition as disclosed herein. Associated with such container(s) can be various written materials such as instructions for use, or a notice in the form prescribed by a governmental agency regulating the manufacture, use or sale of pharmaceuticals or biological products, which notice reflects approval by the agency of manufacture, use, or sale for human administration.

As there may be advantages to mixing a component of a composition described herein and a pharmaceutically acceptable carrier, excipient or vehicle near the time of use, kits in which components of the compositions are packaged separately are disclosed. For example, the kit can contain an active ingredient in a powdered or other dry form in, for example, a sterile vial or ampule and, in a separate container within the kit, a carrier, excipient, or vehicle, or a component of a carrier, excipient, or vehicle (in liquid or dry form). In one aspect, the kit can contain a component in a dry form, typically as a powder, often in a lyophilized form in, for example, a sterile vial or ampule and, in a separate container within the kit, a carrier, excipient, or vehicle, or a component of a carrier, excipient, or vehicle. Alternatively, the kit may contain a component in the form of a concentrated solution that is diluted prior to administration. Any of the components described herein, any of the carriers, excipients or vehicles described herein, and any combination of components and carriers, excipients or vehicles can be included in a kit.

Optionally, a kit may also contain instructions for preparation or use (e.g., written instructions printed on the outer container or on a leaflet placed therein) and one or more devices to aid the preparation of the solution and/or its administration to a patient (e.g., one or a plurality of syringes, needles, filters, tape, tubing (e.g., tubing to facilitate intravenous administration) alcohol swabs and/or the Band-Aid® applicator). Compositions which are more concentrated than those administered to a subject can be prepared. Accordingly, such compositions can be included in the kits with, optionally, suitable materials (e.g., water, saline, or other physiologically acceptable solutions) for dilution. Instructions included with the kit can include, where appropriate, instructions for dilution.

In other embodiments, the kits can include pre-mixed compositions and instructions for solubilizing any precipitate that may have formed during shipping or storage. Kits containing solutions of Compound I, or a pharmaceutically acceptable salt thereof, and one or more carriers, excipients or vehicles may also contain any of the materials mentioned above (e.g., any device to aid in preparing the composition for administration or in the administration per se). The instructions in these kits may describe suitable indications (e.g., a description of patients amenable to treatment) and instructions for administering the solution to a patient.

### EXAMPLES

### Methods

All outcome measures were evaluated by individuals who were blind to the experimental group or individual manipulation.

Animals

C57BL/6J (Jackson laboratory), Vglut2-Cre¹⁰, Vgat-Cre¹⁰, Chat-Cre¹⁷ and CAG-lox-CAT-lox-EGFP (cc-EGFP¹¹) mice (female, 8 weeks old) were used. Mutants were backcrossed with C57/BL/6J strain at least 3 generations. Mice were maintained in a pathogen-free environment in accordance with protocols approved by the Institutional Animal Care and Use Committee of the Cincinnati Children's Hospital Medical Center.

Total numbers of mice used for all studies in this report were: 116 C57BL/6J mice, 36 Vglut2-Cre mice (including wild-type littermate and Vglut2-Cre; cc-EGFP mice), 22 Chat-Cre mice (including wild-type littermate and Chat-Cre; cc-EGFP mice), and 8 Vgat-Cre mice (including Vgat-Cre; cc-EGFP mice).

PRV Tracing

Bartha strain PRV152 (expressing GFP; 4.9×10⁹ pfu/ml⁷) and PRV614 (expressing RFP; 3.9×10⁹ pfu/ml¹⁸; gifts from Dr. Lynn Enquist, Princeton University) were used. Under abdominal surgery with anesthesia, PRV was injected into the spleen (10 sites, total 10 μl). Muscle and skin were then sutured. Animals were kept for indicated days and processed for histological analyses.

Spinal Cord Injury

Adult female mice (8 weeks of age) were anesthetized with isoflurane. A laminectomy was performed to expose the spinal cord and complete transection was performed at T3 or T9 level with iridectomy scissors and aspirator as previously described′. The muscle layers and skin on the back were then sutured and mice were injected with 1 ml of saline subcutaneously. They were placed in HEPA filtered cages on the slide warmer at 37° C., under 12 hr of light-dark cycle and fed commercial pellets and water ad libitum.

Dehydration was monitored daily and manual bladder expression was conducted 2× daily. A total of 105 mice received SCI. Of these, seven mice died of unknown causes before intended date of analyses and were therefore excluded from the experiments.

Fluorogold Tracing

One hundred μl of 0.4% FG in distilled water (Fluorochrome) was injected intraperitoneally 7 days before the perfusion.

Immunohistochemistry

The animals were perfused transcardially with 4% paraformaldehyde (PFA). Spinal cords were dissected and post-fixed in the same fixatives overnight at 4° C. The tissues were then cryopreserved in 30% sucrose in PBS overnight then embedded in Tissue-Tek OCT compound (Sakura Finetek). Fifty-μm-thick serial sections (for FIG. 1, f-q, FIGS. 4 and 5) or 20-μm-thick of every other section (for the other experiments) were made with a cryostat and mounted on Superfrost Plus slides (Fisher).

Sections for synapse counting (FIG. 6) were incubated in PBS overnight after fixation, embedded in low-melting agarose gel then 80-μm-thick of floating sections were made with a vibrotome (Leica).

For immunohistochemistry, sections were blocked with 1% bovine serum albumin (BSA)/0.3% Triton X-100 in PBS for 2 hr and then incubated with the following primary antibodies overnight at 4° C.: sheep anti-GFP (1:1000; AbD Serotec), rabbit anti-GFP (1:1000; Invitrogen), rat anti-GFP (1:1000; Nacalai), rabbit anti-RFP (Rockland, 1:1000), rabbit anti-DsRed (1:500, Clontech), goat anti-choline acetyltransferase (Chat; 1:100; Millipore), rat anti-CD45R (B220)-FITC (1:20; BD Biosciences), rabbit anti-fluorogold (1:500, Fluorochrome), guinea pig anti-vGlut2 (1:10000, Millipore), and goat anti-c-Fos antibodies (1:100, Santa cruz). After washing with 0.1% Tween20/PBS, the sections were incubated with corresponding secondary antibodies: Alexa Fluor 488, 568, 647 or DyLight 549 donkey anti-sheep, rabbit, rat, guinea pig or goat IgG (1:1000; Invitrogen or Jackson ImmunoResearch) for 2 hr at room temperature. For labeling vibrotome sections, tissues were incubated with primary or secondary antibody 3 overnights at 4° C. Images were acquired using a wide-field fluorescence (Zeiss, AXIO IMAGER Z1) or confocal microscope (Nikon AIR⁺).

Quantification of PRV-Positive Neurons

To evaluate the normal neural-splenic circuit, brains and spinal cords were analyzed at 48 (n=2), 72 (n=2), 96 (n=2), 108 (n=1), 120 (n=2), 144 (n=3) or 168 hr (n=2) after PRV injection (FIG. 4, TABLE 1). Specific regions of the brain were identified and mapped using the atlas by Paxinos and Franklin (2001)¹⁹. To verify that retrograde PRV labeling from the spleen occurs via the splenic nerve and not as a result of spurious labeling due to blood-borne virus, the splenic nerve was cut before PRV injection then analyzed at 120 hr (n=2). For counting in the spinal cord, PRV-positive cells were counted then analyzed in horizontal spinal cord sections at 96 hr after PRV injection (FIG. 1f-t, FIG. 5). Pilot studies revealed that this time point allowed consistent labeling in SPNs and presumptive interneurons without causing overt degeneration in PRV⁺ neurons (FIG. 4, e-g, TABLE 1). Moreover, in these mice, robust labeling was evident in neuron clusters over multiple spinal segments. The total number of PRV⁺ neurons was counted in each sub-region of the spinal cord; the left, the right, the medial zone which is along the central canal, the intermediate zone which is the central part of the gray matter, and the lateral zone which is at the interface between gray matter and white matter (see FIG. 1h). Most animals showed left side-dominant distribution (ipsilateral to PRV injection into spleen), but some animals showed an opposite tendency. Hence the quantification was done separately as ipsilateral and contralateral sides (FIG. 1r-t). The number of spinal cords analyzed were: T1-3, n=4; T4-9, n=5; T10-13, n=4; L1-5, n=5; S1-2, n=5; for each control, T9 SCI, or T3 SCI group.

Quantification of FG⁺ and Vglut2-GFP⁺, Vgat-GFP⁺, Chat⁺ neurons were done in horizontal sections at T4-9 spinal level. The number of spinal cords analyzed was: control, n=4; T9 SCI, n=4; T3 SCI, n=4 for FG and PRV double injection experiments (FIGS. 2a-m): n=3 each for characterizing interneuron subtype (FIGS. 2n-z). In ˜30% of mice (25/91), Applicant observed no labeling or very few PRV⁺ cells (˜10 cells in the lateral edge of the gray matter) in the thoracic cord. This variability was not specific to any experimental group and was considered an intrinsic variability in injection and/or uptake of the virus. Samples from these mice were excluded from further analyses.

Quantification of vGlut2⁺ Presynaptic Puncta on SPNs

Horizontal floating sections (T4-9 level) were stained with rabbit anti-FG, sheep anti-GFP, and guinea pig anti-vGlut2 antibodies (80-μm-thick) then were mounted on slides and coverslipped. Z-stack images were acquired with confocal microscopy. FG⁺/PRV-GFP⁺ SPNs with their entire cell body localized within the plane of section and not overlapped with other cells were selected and 3D images were reconstructed using Imaris software (BITPLANE). VGlut2⁺ puncta on the surface of GFP⁺ SPN soma were manually selected and the density (vGlut2⁺ puncta number/GFP⁺ surface) was calculated. The number of animals analyzed were: controls without injury, n=2 (44 cells); day 7 after T3 SCI, n=3 (29 cells); day 10, n=1 (10 cells); day 28, n=5 (67 cells). Data of day 7 and 10 were pooled into one group as a time interval that precedes post-SCI immune suppression′.

PACT Clearing of Spleen

Spleens were cleared using the passive CLARITY (PACT) protocol²⁰. Harvested spleens were fixed in 4% PFA overnight and washed in PBS. The samples were incubated overnight at 4° C. in A4P0 hydrogel monomer solution (4% acrylamide and 0% PFA in PBS), containing 0.25% photoinitiator 2,2′-Azobis[2-(imidazolin-2-yl)propane]dihydrochloride (VA-044, Wako). To polymerize the tissue-hydrogel matrix, samples were then incubated in a 37° C. oven with shaker for 2-3 hr. Polymerized tissues were washed with PBS and then transferred to a clearing solution containing 8% SDS diluted in 0.1M PBS and incubated at 37° C. for 5 days with shaking. The samples were washed with 5 changes of PBS over 1 day and were incubated in blocking solution containing 10% normal donkey serum, 0.1% triton X-100 and 0.01% sodium azide at room temperature for 24 hr with shaking. They were then incubated with rabbit anti-tyrosine hydroxylase (TH) antibody (1:500, Millipore) in blocking solution at 4° C. for 4 days. They were washed with 5 changes of PBS over 1 day and incubated with Alexa 568 donkey anti-rabbit IgG antibody (1:500, Invitrogen) in blocking solution at 4° C. for 2 days. They were washed with 5 changes of PBS over 1 day and incubated in RIMS solution (88% Histodenz, 0.01% Sodium azide in 0.02M phosphate buffer, pH to 7.5) for 24 hr. Images were acquired using a multiphoton confocal microscope (Nikon, AlMP).

Colorectal Distension (CRD) and c-Fos Analyses

Colorectal distension was induced as previously reported3, 12, 13 with minor modification. A 4-French, 60 mm balloon-tipped catheter (Swan-Ganz monitoring catheter model 116F4; Edwards Life Sciences) was inserted 1.5 cm into the rectum before securing the catheter to the tail with surgical tape. The balloon was inflated with 0.2 ml of air for 30 sec then deflated 60 sec. This 90 sec cycle was repeated for 90 min. Mice were perfused 30 min after the final cycle of inflation. The number of c-Fos⁺ cells were counted and analyzed in 2 representative horizontal spinal cord sections at T4-9 level from multiple SCI animals with or without CRD stimulation. The number of animals analyzed were: uninjured with or without CRD, n=3/group; T3 SCI (day 28) without CRD, n=3; T3 SCI (day 28) with CRD, n=5; Vglut2-Cre; cc-EGFP+T3 SCI (day 28) with CRD, n=3; Chat-Cre; cc-EGFP+T3 SCI (day 28) with CRD, n=3 (FIG. 7). To evaluate the effects of DREADD in mice with CRD-induced activation of visceral-sympathetic reflex, CNO (1.0 mg/kg body weight, i.p.; Sigma) or vehicle saline was injected into Vglut2-Cre+T3SCI+AAV8-hSyn-DIO-hM4D(Gi)-mCherry mice at day 28 post-injury, 30 min before the induction of CRD (n=3 each) (FIG. 9).

DREADD Experiment

AAV8-hSyn-DIO-hM4Di-mCherry (AAV8-hSyn-DIO-hM4D(Gi)-mCherry; 7×10¹² vg/ml; UNC Vector Core) was injected into T5 and 7 levels of the spinal cord of Vglut2-Cre, Chat-Cre, or WT mice at P14 (0.8 μl/site, 0.25 mm lateral, 0.5 mm in depth). Applicant confirmed that AAV-mediated transduction was restricted to neurons within and adjacent to the injection site and virus did not get retrogradely transported into brain stem (FIG. 8, j). T3 SCI was induced at 8 weeks old, and then CNO (1.0 mg/kg body weight, i.p.; Sigma) was injected twice per day from day 14 to 27 post-injury (FIG. 3b). Small cohorts of mice from each colony were used as they reached the appropriate age for experimentation. Total number of mice used across different experiments were WT control, 3 separate rounds (n=3, 3, 3, total n=9); WT+T3 SCI, 5 separate rounds (n=2, 1, 2, 2, 1, total 8); WT+T3 SCI+AAV8-hSyn-DIO-hM4D(Gi)-mCherry+CNO, 3 rounds (n=4, 2, 2, total 8); Vglut2-Cre+T3 SCI+AAV8-hSyn-DIO-hM4D(Gi)-mCherry+CNO, 5 rounds (n=2, 1, 1, 2, 2, total 8); Chat-Cre+T3 SCI+AAV8-hSyn-DIO-hM4D(Gi)-mCherry+CNO, 4 rounds (n=4, 1, 1, 2, total 8).

In some experiments, spleens were fixed via immersion in 4% PFA fixation (i.e., no flow cytometry analyses; WT control, n=4; WT+T3 SCI, n=5; WT+T3 SCI+AAV8-hSyn-DIO-hM4D(Gi)-mCherry+CNO, n=4; Vglut2-Cre+T3 SCI+AAV8-hSyn-DIO-hM4D(Gi)-mCherry+CNO, n=4; Chat-Cre+T3 SCI+AAV8-hSyn-DIO-hM4D(Gi)-mCherry+CNO, n=6).

Female C57BL/6J mice were used for WT control and WT+T3 SCI group, and WT littermate with Vglut2-Cre and Chat-Cre mice (n=4, each) were used for WT+T3 SCI+AAV8-hSyn-DIO-hM4D(Gi)-mCherry+CNO group.

Flow Cytometry

Spleens were harvested in Flow Cytometry Staining Buffer (eBioscience) and cell suspensions were made by expressing spleen tissue through a cell strainer using the plunger of a 10 ml syringe. Cell suspensions were then washed, centrifuged and resuspended in 1 ml of the buffer. Splenocytes were counted on a hemocytometer and 5×10⁵ cells were incubated with Fc-block (anti-mouse-CD16/CD32 antibody; eBioscience). Samples were stained with the following antibodies: anti-CD45R (B220)-FITC (clone RA3-6B2; BD Biosciences), anti-CD4-FITC (clone GK1.5; BD Biosciences), anti-CD8-PE (clone 53-6.7; BD Biosciences), and isotype-specific control anti-rat IgG2a κ-FITC and -PE antibodies (clone R35-95; BD Biosciences). All antibodies were diluted to 0.5 μg for 5×10⁵ cells. Splenocytes (10,000 events/sample) were analyzed using a FACSCanto flow cytometer with FACSDiva software (BD Biosciences). Data analyses were conducted with FlowJo software (FlowJo). Dot plots of forward scatter (FSC) vs side scatter (SSC) were used to exclude debris, and then doublets were excluded using FSC-height (H) vs FSC-width (W) and SSC-H vs SSC-W plots (FIG. 10, a-i). The number of each subset was calculated as the percentage of labeled cells multiplied by the total number of splenocytes.

Statistical Analysis.

Quantitative data are expressed as the mean±s.e.m. Differences among the groups were analyzed by one-way ANOVA followed by Tukey-Kramer test. Differences between experimental group pairs were analyzed with Student's t test (two tailed). A P-value less than 0.05 was considered significant.

### REFERENCES

1. Blackmer, J. Rehabilitation medicine: 1. Autonomic dysreflexia. CMAJ 169, 931-935 (2003).
2. Inskip, J. A., Ramer, L. M., Ramer, M. S. & Krassioukov, A. V. Autonomic assessment of animals with spinal cord injury: tools, techniques and translation. Spinal Cord 47, 2-35 (2009).
3. Zhang, Y., et al. Autonomic dysreflexia causes chronic immune suppression after spinal cord injury. J Neurosci 33, 12970-12981 (2013).
4. Lucin, K. M., Sanders, V. M., Jones, T. B., Malarkey, W. B. & Popovich, P. G. Impaired antibody synthesis after spinal cord injury is level dependent and is due to sympathetic nervous system dysregulation. Exp Neurol 207, 75-84 (2007).
5. Lucin, K. M., Sanders, V. M. & Popovich, P. G. Stress hormones collaborate to induce lymphocyte apoptosis after high level spinal cord injury. J Neurochem 110, 1409-1421 (2009).
6. Meisel, C., Schwab, J. M., Prass, K., Meisel, A. & Dimagl, U. Central nervous system injury-induced immune deficiency syndrome. Nat Rev Neurosci 6, 775-786 (2005).
7. Smith, B. N., et al. Pseudorabies virus expressing enhanced green fluorescent protein: A tool for in vitro electrophysiological analysis of transsynaptically labeled neurons in identified central nervous system circuits. Proc Natl Acad Sci USA 97, 9264-9269 (2000).
8. Cano, G., Sved, A. F., Rinaman, L., Rabin, B. S. & Card, J. P. Characterization of the central nervous system innervation of the rat spleen using viral transneuronal tracing. J Comp Neurol 439, 1-18 (2001).
9. Anderson, C. R. & Edwards, S. L. Intraperitoneal injections of Fluorogold reliably labels all sympathetic preganglionic neurons in the rat. J Neurosci Methods 53, 137-141 (1994).
10. Vong, L., et al. Leptin action on GABAergic neurons prevents obesity and reduces inhibitory tone to POMC neurons. Neuron 71, 142-154 (2011).
11. Nakamura, T., Colbert, M. C. & Robbins, J. Neural crest cells retain multipotential characteristics in the developing valves and label the cardiac conduction system. Circ Res 98, 1547-1554 (2006).
12. Hou, S., et al. Plasticity of lumbosacral propriospinal neurons is associated with the development of autonomic dysreflexia after thoracic spinal cord transection. J Comp Neurol 509, 382-399 (2008).
13. Duale, H., Lyttle, T. S., Smith, B. N. & Rabchevsky, A. G. Noxious colorectal distention in spinalized rats reduces pseudorabies virus labeling of sympathetic neurons. J Neurotrauma 27, 1369-1378 (2010).
14. Stemson, S. M. & Roth, B. L. Chemogenetic tools to interrogate brain functions. Annu Rev Neurosci 37, 387-407 (2014).
15. Brommer, B., et al. Spinal cord injury-induced immune deficiency syndrome enhances infection susceptibility dependent on lesion level. Brain (2016).
16. Iversen, P. O., et al. Depressed immunity and impaired proliferation of hematopoietic progenitor cells in patients with complete spinal cord injury. Blood 96, 2081-2083 (2000).
17. Rossi, J., et al. Melanocortin-4 receptors expressed by cholinergic neurons regulate energy balance and glucose homeostasis. Cell Metab 13, 195-204 (2011).
18. Banfield, B. W., Kaufman, J. D., Randall, J. A. & Pickard, G. E. Development of pseudorabies virus strains expressing red fluorescent proteins: new tools for multisynaptic labeling applications. J Virol 77, 10106-10112 (2003).
19. Paxinos, G. The mouse brain in stereotaxic coordinates. (Academic press, San Diego, Calif., 2001).
20. Yang, B., et al. Single-cell phenotyping within transparent intact tissue through whole-body clearing. Cell 158, 945-958 (2014).

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating a condition associated with spinal cord injury, comprising the step of administering an agent that inhibits excitatory neurons and/or activates inhibitory interneurons.

2. The method of claim 1, wherein said condition is selected from AD (autonomic dysreflexia), immune suppression, leukocyte apoptosis, splenic atrophy and/or leucopenia.

3. The method of claim 1, wherein said agent is an NMDA (glutamate receptor) antagonist.

4. The method of claim 1, wherein said agent is a a-amino-3-hydroxy-5-methyl-4-isoxazolepropionic acid (AMPA) receptor antagonist.

5. The method of claim 4, wherein said antagonist has the structure of 6-cyano-7-nitroquinoxaline-2,3-dione (CNQX), or a pharmaceutically acceptable salt thereof.

6. The method of claim 1, wherein said agent is NMDA receptor antagonist DL-2-amino-5-phosphonovalerate (DL-APV), or a pharmaceutically acceptable salt thereof.

7. The method of claim 1, wherein said agent is DL-2-amino-4-phosphonobutyric acid (DL-APB), or a pharmaceutically acceptable salt thereof.

8. The method of claim 1, wherein said agent is selected from phencyclidine (PCP), dextromethorphan, ketamine, or a pharmaceutical salt thereof, and combinations thereof.

9. The method of claim 1, wherein said agent is selected from benzodiazepine and THC.

10. The method of claim 1, wherein said agent is a AAV-vGlut2-promoter-DREADD construct.

11. An article of manufacture comprising:
a. a container comprising a label; and
b. a composition comprising an agent that inhibits excitatory neurons and/or activates inhibitory interneurons, wherein the label indicates that the composition is to be administered to an individual having a condition associated with a spinal cord injury.

12. A kit comprising an agent that inhibits excitatory neurons and/or activates inhibitory interneurons and instructions for treating a subject with a condition associated with spinal cord injury with said agent.

13. The kit of claim 12, further comprising a means for delivery of said composition to a human.

14. The kit of claim 12, wherein said composition is formulated for intravenous administration to an individual.
